(19) United States
(12) Patent Application Publication
(10) Pub. No.: US 2007/0198092 A1
(43) Pub. Date: Aug. 23, 2007

(54) System for inserting artificial intervertebral discs

(75) Inventors: Joseph P. Errico, Green Brook, NJ (US); Michael W. Dudasik, Nutley, NJ (US); Rafail Zubok, Midland Park, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: SpineCore, Inc., Summit, NJ (US)

(21) Appl. No.: 11/789,327

(22) Filed: Apr. 24, 2007

Related U.S. Application Data
(63) Continuation of application No. 10/425,267, filed on Apr. 29, 2003, now Pat. No. 7,235,081.
Application No. 10/425,267 is a continuation-in-part of application No. 10/282,356, filed on Oct. 29, 2002, now Pat. No. 7,169,182.
Application No. 10/282,356 is a continuation-in-part of application No. 10/256,160, filed on Sep. 26, 2002, now Pat. No. 6,989,032.
Application No. 10/256,160 is a continuation-in-part of application No. 10/175,417, filed on Jun. 19, 2002.
Application No. 10/175,417 is a continuation-in-part of application No. 10/151,280, filed on May 20, 2002.
Application No. 10/151,280 is a continuation-in-part of application No. 10/140,153, filed on May 7, 2002, now abandoned.
Application No. 10/140,153 is a continuation-in-part of application No. 10/128,619, filed on Apr. 23, 2002, now Pat. No. 6,863,689.
Application No. 10/128,619 is a continuation-in-part of application No. 09/906,119, filed on Jul. 16, 2001, now Pat. No. 6,607,559.
Application No. 10/128,619 is a continuation-in-part of application No. 09/982,148, filed on Oct. 18, 2001, now Pat. No. 6,673,113.

(51) Int. Cl. A61F 2/44; A61F 2/46; A61B 17/92

(52) U.S. Cl. 623/17.14; 606/99

(57) ABSTRACT

A system for stabilizing an intervertebral segment includes an intervertebral implant having a first plate, a second plate, and an articulating joint coupling the first and second plates, whereby at least one of the plates has an inner surface having at least one hole. The system includes an instrument for holding the implant, the system having a shaft with a proximal end, a distal end and a longitudinal axis extending between the proximal and distal ends, a body attached to the distal end of the shaft, and a pin having a hooked end that is extendable from the body for being inserted into the at least one hole of the implant so as to couple the implant with the distal end of the holding instrument.

### CROSS-REFERENCE TO RELATED APPLICATIONS

The present application is a continuation of U.S. patent application Ser. No. 10/425,267, filed Apr. 29, 2003, entitled “Wedge Plate Inserter/Impactor and Related. Methods for Use in Implanting an Artificial Intervertebral Disc,” now allowed, which is a continuation-in-part application of U.S. patent application Ser. No. 10/282,356, filed Oct. 29, 2002, entitled “Instrumentation and Methods For Use In Implanting an Artificial Intervertebral Disc,” which is a continuation-in-part application of U.S. patent application Ser. No. 10/256,160, filed Sep. 26, 2002, entitled “Artificial Intervertebral Disc Having Limited Rotation Using a Captured Ball and Socket Joint With a Solid Ball and Compression Locking Post,” which is a continuation-in-part application of U.S. patent application Ser. No. 10/175,417, filed Jun. 19, 2002, entitled “Artificial Intervertebral Disc Utilizing a Ball Joint Coupling,” which is a continuation-in-part application of U.S. patent application Ser. No. 10/151,280, filed May 20, 2002, entitled “Tension Bearing Artificial Disc Providing a Centroid of Motion Centrally Located Within an Intervertebral Space,” which is a continuation-in-part application of both U.S. patent application Ser. No. 09/970,479, filed Oct. 4, 2001, entitled “Intervertebral Spacer Device Utilizing a Spirally Slotted Belleville Washer Having Radially Extending Grooves,” as well as U.S. patent application Ser. No. 10/140,153, filed May 7, 2002, entitled “Artificial Intervertebral Disc Having a Flexible Wire Mesh Vertebral Body Contact Element,” the former being a continuation-in-part application of U.S. patent application Ser. No. 09/968,046, filed Oct. 1, 2001, entitled “Intervertebral Spacer Device Utilizing a Belleville Washer Having Radially Extending Grooves” and the latter being a continuation-in-part application of both U.S. patent application Ser. No. 09/970,479 (detailed above) as well as U.S. patent application Ser. No. 10/128,619, filed Apr. 23, 2002, entitled “Intervertebral Spacer Having a Flexible Wire Mesh Vertebral Body Contact Element,” which is a continuation-in-part application of both U.S. patent application Ser. No. 09/906,119, filed Jul. 16, 2001, and entitled “Trial Intervertebral Distraction Spacers” as well as U.S. patent application Ser. No. 09/982,148, filed Oct. 18, 2001 and entitled “Intervertebral Spacer Device Having Arch Shaped Spring Elements.” All of the above mentioned applications are hereby incorporated by reference herein in their respective entireties.

### FIELD OF THE INVENTION

This invention relates generally to systems and methods for use in spine arthroplasty, and more specifically to instruments for inserting and impacting artificial intervertebral discs, and methods of use thereof.

### BACKGROUND OF THE INVENTION

The bones and connective tissue of an adult human spinal column consists of more than twenty discrete bones coupled sequentially to one another by a tri-joint complex that consists of an anterior disc and the two posterior facet joints, the anterior discs of adjacent bones being cushioned by cartilage spacers referred to as intervertebral discs. These more than twenty bones are anatomically categorized as being members of one of four classifications: cervical, thoracic, lumbar, or sacral. The cervical portion of the spine, which comprises the top of the spine, up to the base of the skull, includes the first seven vertebrae. The intermediate twelve bones are the thoracic vertebrae, and connect to the lower spine comprising the five lumbar vertebrae. The base of the spine is the sacral bones (including the coccyx). The component bones of the cervical spine are generally smaller than those of the thoracic spine, which are in turn smaller than those of the lumbar region. The sacral region connects laterally to the pelvis. While the sacral region is an integral part of the spine, for the purposes of fusion surgeries and for this disclosure, the word spine shall refer only to the cervical, thoracic, and lumbar regions.

The spinal column is highly complex in that it includes these more than twenty bones coupled to one another, housing and protecting critical elements of the nervous system having innumerable peripheral nerves and circulatory bodies in close proximity. In spite of these complications, the spine is a highly flexible structure, capable of a high degree of curvature and twist in nearly every direction.

Genetic or developmental irregularities, trauma, chronic stress, tumors, and degenerative wear are a few of the causes that can result in spinal pathologies for which surgical intervention may be necessary. With respect to the failure of the intervertebral disc, and the insertion of implants and/or height restorative devices, several methods and devices have been disclosed in the prior art that achieve immobilization and/or fusion of adjacent bones by implanting artificial assemblies in or on the spinal column. More recently, the development of non-fusion implant devices, which purport to permit continued natural movement in the tri-joint complex, have provided great promise as a preferably alternative to fusion devices. The region of the back that needs to be corrected, as well as the individual variations in anatomy, determine the appropriate surgical protocol and implantation assembly. Generally, the preparation of the intervertebral space for the receipt of fusion or non-fusion devices involves removing the damaged disc material and thereafter distracting the adjacent vertebral bones to their appropriate distance apart. Once the proper height of the intervertebral space is restored, the fusion or non-fusion device can be implanted.

It is an object of the invention to provide instrumentation and methods that enable surgeons to more accurately, easily, and efficiently implant fusion or non-fusion devices. Other objects of the invention not explicitly stated will be set forth and will be more clearly understood in conjunction with the descriptions of the preferred embodiments disclosed hereafter.

### SUMMARY OF THE INVENTION

The preceding objects are achieved by the invention, which includes, among other aspects, an inserter/impactor (sometimes referred to herein as an “inserter/impactor”) useful for holding and manipulating artificial intervertebral discs.

More particularly, the systems and methods disclosed herein are intended for use in spine arthroplasty procedures, and specifically for use with the systems and methods described herein in conjunction with the systems and methods in conjunction with the systems and methods described in U.S. patent application Ser. No. 10/282,356 (filed Oct. 29, 2002) entitled “Instrumentation and Methods For Use In Implanting an Artificial Intervertebral Disc” (hereinafter referred to as “the '356 application”) as well as U.S. patent application Ser. No. 10/256,160 (filed Sep. 26, 2002) entitled “Artificial Intervertebral Disc Having Limited Rotation Using a Captured Ball and Socket Joint With a Solid Ball and Compression Locking Post” (hereinafter referred to as “the '160 application”) as well as U.S. patent application Ser. No. 09/906,127 (filed Jul. 16, 2001) entitled “Insertion Tool For Use With Intervertebral Spacers” (hereinafter referred to as ‘the '127 application“), both applications of which are mentioned above. However, it should be understood that the systems and methods described herein are also suitable for use with other systems and methods without departing from the scope of the invention.

While the instrumentation described herein (e.g., the inserter/impactor) will be discussed for use with the artificial intervertebral disc of FIGS. 1a-h of the present application (hereinafter, such figures will merely be referred to as “FIGS. 1a-h”), such discussions are merely by way of example and not intended to be limiting of their uses. Thus, it should be understood that the instrumentation and methods can be used with any of the artificial intervertebral discs disclosed in the '356 or '160 applications, or any other artificial intervertebral disc having (or being modifiable or modified to have) suitable features therefor. Moreover, it is anticipated that the features of the artificial intervertebral discs (e.g., plate surfaces and engagement holes) that are used by the inserter/impactor discussed herein to hold and/or manipulate the artificial intervertebral disc can be applied, individually, or collectively or in various combinations, to other trials, spacers, artificial intervertebral discs, or other orthopedic devices as stand-alone innovative features for enabling such trials, spacers, artificial intervertebral discs, or other orthopedic devices to be more efficiently and more effectively held and/or manipulated by the inserter/impactor described herein or by tools having suitable features. In addition, it should be understood that the invention encompasses instrumentation and methods for implanting artificial intervertebral discs, spacers, trials (static or dynamic), and/or other orthopedic devices, that have one or more of the features disclosed herein, in any combination, and that the invention is therefore not limited to artificial intervertebral discs, spacers, trials, and/or other orthopedic devices having all of the features simultaneously.

Preferably, with regards to each artificial intervertebral disc to be implanted, a plurality of sizes of the artificial intervertebral disc would be available (e.g., the artificial intervertebral disc 160 of FIGS. 1a-h). That is, preferably, a plurality of the same type of artificial intervertebral disc would be available, each of the plurality having a respective width and depth dimension combination that allows it to fit within a correspondingly dimensioned intervertebral space. For example, the plurality of artificial intervertebral discs could include artificial intervertebral discs having widths being either 35 mm or 40 mm, and depths ranging from 14 mm to 18 mm in 1 mm increments, for a total of 10 discs. It should be understood that the artificial intervertebral discs can be offered in a variety of dimensions without departing from the scope of the invention, and that the dimensions specifically identified and quantified herein are merely exemplary. Each of the plurality of artificial intervertebral disc preferably further includes features that can be used by the inserter/impactor (described below) and/or the inserter/impactor described in the '356 application.

With regard to features that can be used by the inserter/impactor described in the '356 application, each artificial intervertebral disc includes an anteriorly facing flat surface, flanked by two anteriolaterally facing flat surfaces (one on each side of the anteriorly facing flat surface), and, to provide for holding of the disc for an anterior insertion approach, a hole spaced from the anteriorly facing flat surface, the hole having a longitudinal axis parallel to the anteriorly facing flat surface. The holding pin of the inserter/impactor fits within the hole, and the angled flat surfaces of the disc fit against the correspondingly angled flat surfaces of the inserter/impactor, and operation of the inserter/impactor pulls the holding pin toward the flat surface of the inserter/impactor opposite the pin, to rigidly hold the disc by the lower baseplate. The holding pin protrudes from the wedge-shaped extended surface of the distal end of the inserter/impactor and is restricted from upward movement with respect to the distal head by the presence of the wedge-shaped extended surface of the distal end of the inserter/impactor. More particularly, with any attempted upward movement of the holding pin, the pin encounters the upper surface of the channel in which the pin travels, preventing any such upward movement. When the intervertebral disc is held in this manner, rotation of the disc about a longitudinal axis relative to the inserter/impactor is prevented by interference of the corners of the disc's flat surfaces and the corners of the inserter/impactor's flat surfaces, similar to the manner in which a wrench holding a nut prevents rotation of the nut relative to the wrench. Further, when the disc is held in this manner, rotation of the disc about a lateral axis of the disc relative to the inserter/impactor is prevented by interference of the inwardly facing surface of the first baseplate (e.g., upper baseplate) of the disc and the corresponding surface (e.g., upper surface) of the wedge on the distal end, and by interference of the inwardly facing surface of the second baseplate (e.g., lower baseplate) of the disc and the corresponding surface (e.g., lower surface) of the wedge on the distal end. It is preferable that the wedge on the inserter/impactor will interfere between the first and second baseplates (e.g., upper and lower) so that the surfaces of the first and second baseplates align at a preferred 15 degrees angle of lordosis when the disc is held by the inserter/impactor.

Preferably, in order to provide for a holding of the disc for two additional (here, anteriolateral) insertion approaches, each disc also include two additional holes, one spaced apart from one of the anteriolaterally facing flat surfaces, and the other spaced apart from the other of the anteriolaterally facing flat surfaces. Accordingly, operation of the inserter/impactor can fit the holding pin into either of these two additional holes, and hold the anteriolaterally facing flat surface (the one associated with the hole into which the pin is fit) of the disc against the flat surface of the inserter/impactor opposite the pin. It should be understood that preferably, in order to facilitate these two additional approaches, the angle separating the anteriorly facing flat surface of the disc and one of the anteriolaterally facing flat surfaces of the disc is equal to the angle separating the anteriorly facing flat surface and the other of the anteriolaterally facing flat surfaces.

### BRIEF DESCRIPTION OF THE DRAWINGS

FIGS. 1a-h of the present application show front (FIG. 1a), side cutaway (FIG. 1b), top (FIG. 1c), perspective cutaway (FIG. 1d), bottom cutaway (FIG. 1e), top cutaway (FIG. 1f), bottom perspective (FIG. 1g), and top perspective (FIG. 1h) views of an exemplary artificial intervertebral disc for use with the present invention.

FIGS. 2a-c show side (FIG. 2a), perspective (FIG. 2b), and close-up perspective (FIG. 2c) views of a wedge plate inserter/impactor of the present invention.

FIGS. 3a-d show bottom (FIG. 3a), side (FIG. 3b), top (FIG. 3c), and side cutaway (FIG. 3d) views of a distal end of a wedge plate inserter/impactor of the present invention.

FIGS. 4a-b show top (FIG. 4a) and side (FIG. 4b) views of a wedge plate inserter/impactor of the present invention holding an exemplary artificial intervertebral disc.

FIGS. 4c-e show top (FIG. 4c), side (FIG. 4d), and side cutaway (FIG. 4e) views of a distal end of a wedge plate inserter/impactor of the present invention holding an exemplary artificial intervertebral disc.

### DETAILED DESCRIPTION OF THE PREFERRED EMBODIMENTS

While the invention will be described more fully hereinafter with reference to the accompanying drawings, it is to be understood at the outset that persons skilled in the art may modify the invention herein described while achieving the functions and results of the invention. Accordingly, the descriptions that follow are to be understood as illustrative and exemplary of specific structures, aspects and features within the broad scope of the invention and not as limiting of such broad scope. Like numbers refer to similar features of like elements throughout.

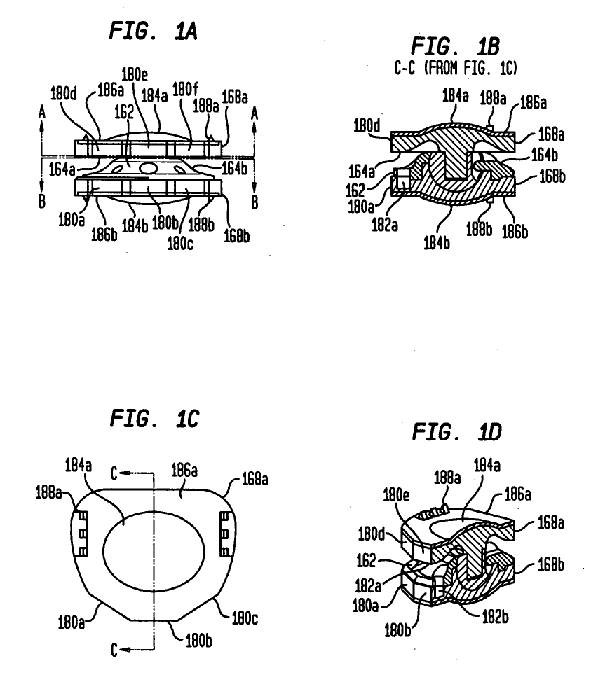

A preferred embodiment of an artificial intervertebral disc (e.g., artificial intervertebral disc 160) for use with the instrumentation of the present invention is referenced and described in the '356 application, and the same description is hereby incorporated by reference herein. The artificial intervertebral disc illustrated in FIGS. 1a-h of the present application is discussed herein with reference to such figures, as an example of an artificial intervertebral disc suitable for use with the present invention.

Referring now to FIGS. 1a-h, an artificial intervertebral disc of the present invention is shown in front (FIG. 1a), side cutaway (FIG. 1b), top (FIG. 1c), side cutaway (FIG. 1d), bottom cutaway (FIG. 1e), top cutaway (FIG. 1f), bottom perspective (FIG. 1g), and top perspective (FIG. 1h) views.

It should be understood that the illustration and reference herein to the artificial intervertebral disc shown in FIGS. 1a-1h is merely to show an example of one type of artificial intervertebral disc that is contemplated by, encompassed by, and suitable for use with, the present invention, and that such illustration and reference herein is not meant to limit the scope of the present invention or limit the uses of the present invention. Rather, any other artificial intervertebral disc (or any other orthopedic device) having suitable features for being manipulated by the instrumentation and methods described herein are contemplated by the present invention. Indeed, the features suitable for manipulation (e.g., angled flat surfaces with adjacent holes) are encompassed by the present invention, regardless of to what orthopedic device they may be applied. Other exemplary suitable artificial intervertebral discs include, but are not limited to, the artificial intervertebral discs described in the '160 application. The artificial intervertebral disc shown in FIGS. 1a-1h has features similar to those of these other suitable artificial intervertebral discs of the '160 application, and it should be understood that such similar features are structurally and functionally as described in the '160 application. Such similar features include an inwardly facing surface of the upper baseplate 164a, and a convex structure 162 on the lower baseplate 168b, the convex structure 162 having an inwardly facing surface 164b.

And, while the instrumentation described herein will be discussed for use with the artificial intervertebral disc of FIGS. 1a-1h, such discussions are merely by way of example and are not intended to be limiting of their uses. Thus, it should be understood that the tools can be used with any of the artificial intervertebral discs disclosed in the '160 application, or any other artificial intervertebral disc having (or being modifiable or modified to have) suitable features therefor. Moreover, it is anticipated that the features of the artificial intervertebral disc (e.g., the flat surfaces and accompanying holes) and/or the static trials (e.g., the cylindrical trunks and flat surfaces and accompanying holes) that are used by the tools discussed herein to hold and/or manipulate these devices can be applied, individually or collectively or in various combinations, to other trials, spacers, artificial intervertebral discs or other orthopedic devices as stand-alone innovative features for enabling such trials, spacers, artificial intervertebral discs, or other orthopedic devices to be more efficiently and more effectively held and/or manipulated by the tools described herein or by other tools having similar features. In addition, it should be understood that the invention encompasses artificial intervertebral discs, spacers, trials (static or dynamic), and/or other orthopedic devices, that have one or more of the features disclosed herein, in any combination, and that the invention is therefore not limited to artificial intervertebral discs, spacers, trials, and/or other orthopedic devices having all of the features simultaneously.

A plurality of static trials are provided primarily for use in determining the appropriate size of an artificial intervertebral disc to be implanted (or whether a particular size of the artificial intervertebral disc can be implanted) into the distracted intervertebral space (e.g., the artificial intervertebral disc 160 of FIGS. 1a-1h). Preferably, for each artificial intervertebral disc to be implanted, a plurality of sizes of the artificial intervertebral disc would be available. That is, preferably, a plurality of the same type of artificial intervertebral disc would be available, each of the plurality having a respective width and depth dimension combination that allows it to fit within a correspondingly dimensioned intervertebral space. For example, the plurality of artificial intervertebral discs could include artificial intervertebral discs having widths being either 35 mm or 40 mm, and depths ranging from 14 mm to 18 mm in 1 mm increments, for a total of 10 discs. Accordingly, preferably, each of the plurality of static trials for use with a particular plurality of differently sized artificial intervertebral discs would have a respective width and depth dimension set corresponding to the width and depth of a respective one of the plurality of differently sized artificial intervertebral discs. For example, the plurality of static trials for use with the set of artificial intervertebral discs described for example could include static trials having widths being either 35 mm or 40 mm, and depths ranging from 14 mm to 18 mm in 1 mm increments, for a total of 10 static trials. It should be understood that the artificial intervertebral discs and/or the static trials can be offered in a variety of dimensions without departing from the scope of the invention, and that the dimensions specifically identified and quantified herein are merely exemplary. Moreover, it should be understood that the set of static trials need not include the same number of trials for each artificial intervertebral disc in the set of artificial intervertebral discs, but rather, none, one, or more than one trial can be included in the trial set for any particular artificial intervertebral disc in the set.

A preferred embodiment of a wedge plate inserter/impactor of the present invention will now be described.

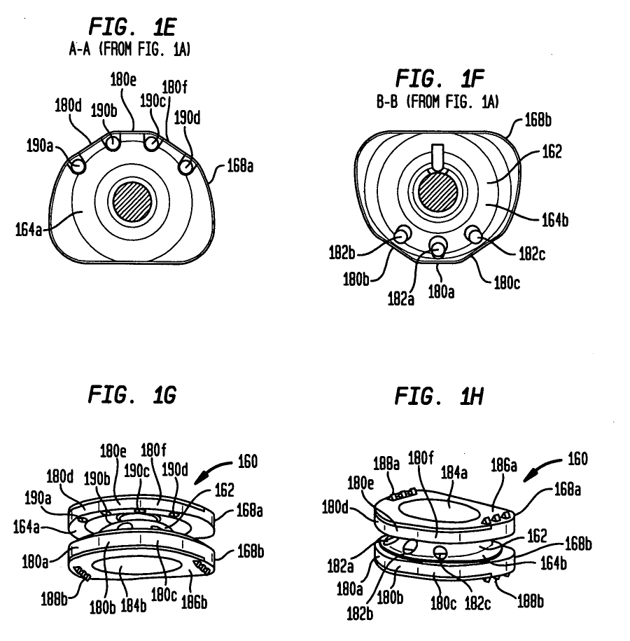

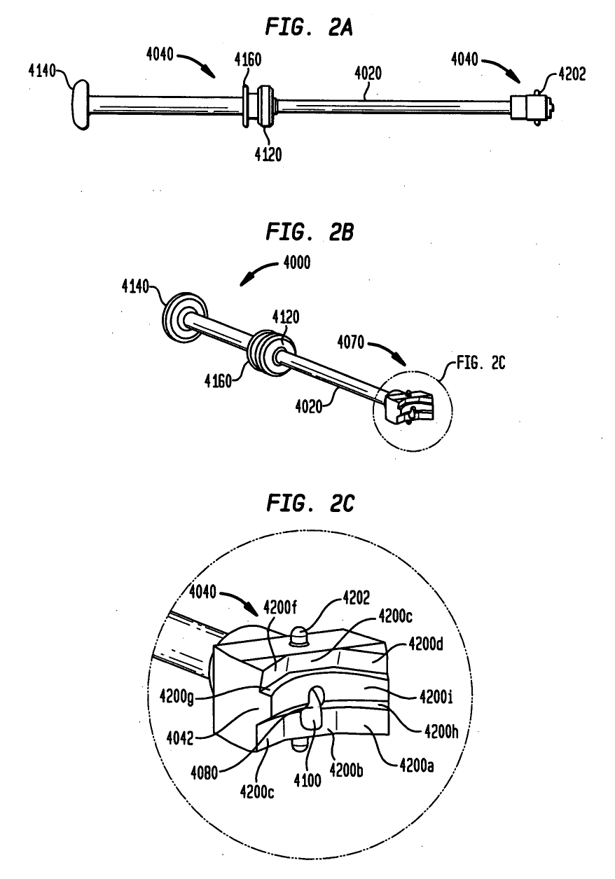

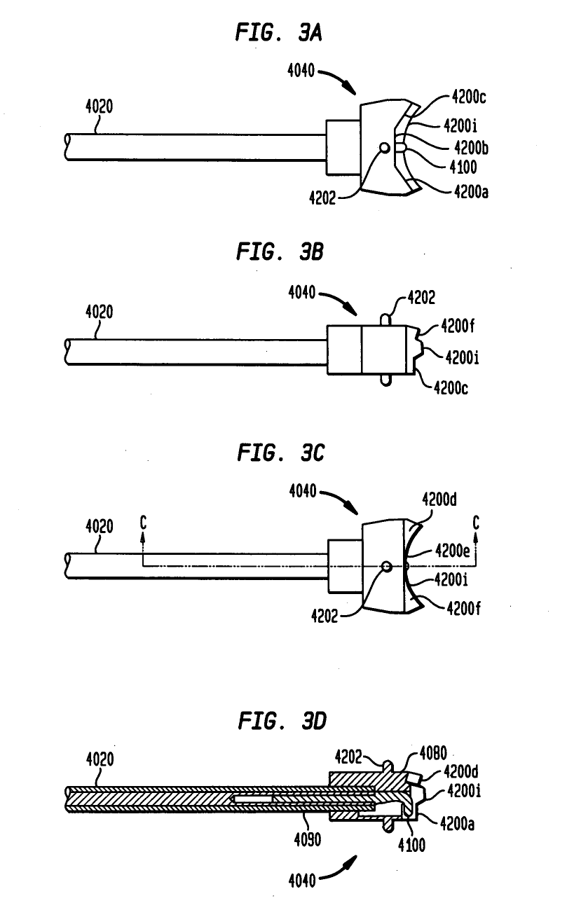

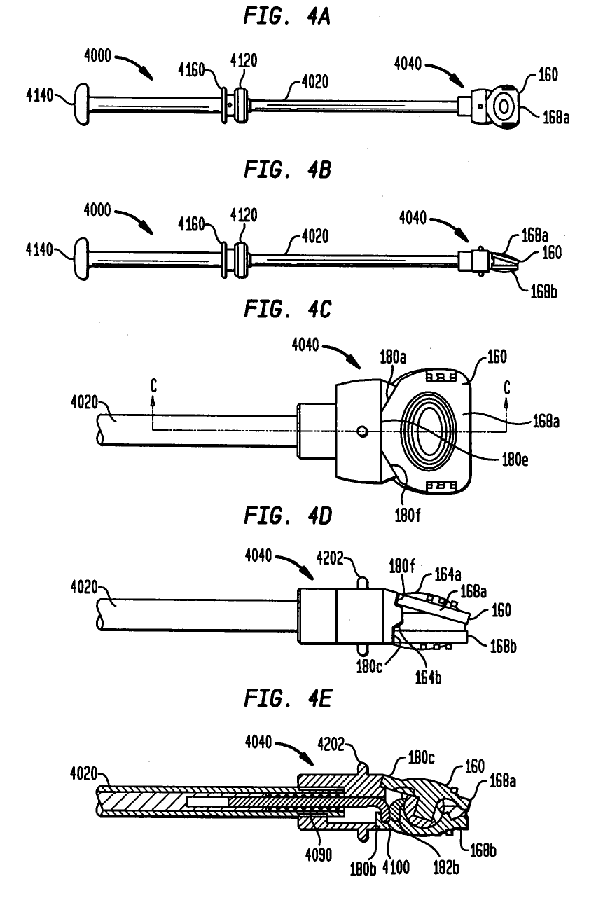

Referring now to FIGS. 2a-4e, FIGS. 2a-c side (FIG. 2a), perspective (FIG. 2b), and close-up perspective (FIG. 2c), and perspective (FIG. 4d) views of a wedge plate inserter/impactor of the present invention. FIGS. 3a-d show bottom (FIG. 3a), side (FIG. 3b), top (FIG. 3c), and side cutaway (FIG. 3d) views of a distal end of a wedge plate inserter/impactor of the present invention. FIGS. 4a-b show top (FIG. 4a) and side (FIG. 4b) views of a wedge plate inserter/impactor of the present invention holding an exemplary artificial intervertebral disc. FIGS. 4c-e show top (FIG. 4c), side (FIG. 4d), and side cutaway (FIG. 4e) views of a distal end of a wedge plate inserter/impactor of the present invention holding an exemplary artificial intervertebral disc.

It should be understood that the illustration and reference herein to the artificial intervertebral disc shown in FIGS. 1a-h of the present application is merely to show an example of one type of artificial intervertebral disc that is contemplated by, encompassed by, and suitable for use with, the present invention, and that such illustration and reference herein is not meant to limit the scope of the present invention or limit the uses of the present invention. Rather, any other artificial intervertebral disc (or any other orthopedic device) having suitable features for being used with the instrumentation and methods described herein are contemplated by the present invention. Indeed, the features suitable for manipulation (e.g., angled flat surfaces with adjacent holes and/or opposing notches, and/or inwardly facing baseplate surfaces) are encompassed by the present invention, regardless of to what orthopedic device they may be applied. Other exemplary suitable artificial intervertebral discs include, but are not limited to, the artificial intervertebral discs described in the '160 application with regard to FIGS. 8a-y, 9a-t, 10a-t, 11a-j, and 12a-o thereof and by the accompanying descriptions therefor (e.g., embodiments identified as the first, second, third, fourth, and fifth preferred embodiments of the fourth embodiment family, etc.). It should be noted that, as can be seen from FIGS. 1a-h of the present application, that the artificial intervertebral disc shown in FIGS. 1a-h of the present application has features similar to those of these other suitable artificial intervertebral discs of the '160 application, and it should be understood that such similar features are structurally and functionally as described in the '160 application. Such similar features include an inwardly facing surface of the upper baseplate, and a convex structure on the lower baseplate, the convex structure having an inwardly facing surface.

And, while the instrumentation described herein (e.g., the inserter/impactor) as well as the instrumentation described in the '356 application (e.g., the inserter/impactor described therein) will be discussed for use with the artificial intervertebral disc of FIGS. 1a-h of the present application, such discussions are merely by way of example and not intended to be limiting of their uses. Thus, it should be understood that the tools can be used with any of the artificial intervertebral discs disclosed in the '356 application or the '160 application, or any other artificial intervertebral disc having (or being modifiable or modified to have) suitable features therefor. Moreover, it is anticipated that the features of the artificial intervertebral disc (e.g., the angled flat surfaces and the inwardly facing baseplate surfaces, and accompanying holes) that are used by the tool discussed herein (or in the '356 application) to hold and/or manipulate these devices (certain features, it should be noted, were first shown and disclosed in the '160 application, the '127 application, and/or the '356 application) can be applied, individually or collectively or in various combinations, to other trials, spacers, artificial intervertebral discs or other orthopedic devices as stand-alone innovative features for enabling such trials, spacers, artificial intervertebral discs, or other orthopedic devices to be more efficiently and more effectively held and/or manipulated by the tools described herein (or in the '356 application) or by other tools having suitable features. In addition, it should be understood that the invention encompasses artificial intervertebral discs, spacers, trials (static or dynamic), and/or other orthopedic devices, that have one or more of the features disclosed herein (or in the '356 application), in any combination, and that the invention is therefore not limited to artificial intervertebral discs, spacers, trials, and/or other orthopedic devices having all of the features simultaneously.

Preferably, for each artificial intervertebral disc to be implanted, a plurality of sizes of the artificial intervertebral disc would be available. That is, preferably, a plurality of the same type of artificial intervertebral disc would be available, each of the plurality having a respective width and depth dimension combination that allows it to fit within a correspondingly dimensioned intervertebral space. For example, the plurality of artificial intervertebral discs could include artificial intervertebral discs having widths being either 35 mm or 40 mm, and depths ranging from 14 mm to 18 mm in 1 mm increments, for a total of 10 discs.

The inserter/impactor 4000 is provided primarily for holding, inserting, repositioning, removing, impacting, extracting, and otherwise manipulating an artificial intervertebral disc having features suitable for being manipulated by the inserter/impactor. (However, it can also be used to hold, insert, reposition, remove, impact, extract, and otherwise manipulate any other orthopedic device having suitable features therefor. For example, it should be understood that distraction of an intervertebral space can be accomplished in conjunction with a cooperating tool or spacer that can be gripped by the inserter/impactor.) Exemplary suitable artificial intervertebral discs include, but are not limited to, the artificial intervertebral disc 160 described herein and the artificial intervertebral discs described in the '160 application with regard to FIGS. 8a-y, 9a-t, 10a-t, 11a-j, and 12a-o thereof and by the accompanying descriptions therefor (e.g., embodiments identified as the first, second, third, fourth, and fifth preferred embodiments of the fourth embodiment family, etc.). Regarding the features suitable for being manipulated by the inserter/impactor 4000, such features include those discussed above as being suitable features on the disc 160, namely, an anteriorly facing flat surface on the second (e.g., lower) baseplate of the trial or disc, flanked by two anteriolaterally facing flat surfaces (one on each side of the anteriorly facing flat surface), and, to provide for holding of the trial or disc for an anterior insertion approach, a hole spaced from the anteriorly facing flat surface, the hole having a longitudinal axis parallel to the anteriorly facing flat surface. Further regarding the features suitable for being manipulated by the inserter/impactor, such features further include the inwardly facing surfaces of the baseplates of the disc.

More particularly, the inserter/impactor 4000 includes a shaft 4020 having a distal end 4040 that has angled flat surfaces 4200a-c corresponding to and fittable against angled flat surfaces of the artificial intervertebral disc (e.g., the surfaces 180a-c of the artificial intervertebral disc 160) to be implanted. The distal end 4040 has angled flat surfaces 4200d-f corresponding to and fittable against angled flat surfaces of the artificial intervertebral disc (e.g., the surfaces 180d-f of the artificial intervertebral disc 160) to be implanted. The distal end 4040 has a wedge-shaped extension 4042 including upper 4200g and lower 4200h wedge surfaces corresponding to and fittable against the inwardly facing surfaces of the artificial intervertebral disc (e.g., the lower surface 164a of the upper baseplate 168a of the disc 160, and the upper surface 164b of the lower baseplate 168b of the disc 160, respectively) to be implanted. For example, in an anterior approach for the disc 160 (as shown in FIGS. 4a-e), 180a and 180d facing 4200a and 4200d, 180b and 180e facing 4200b and 4200e, 180c and 180f facing 4200c and 4200f, and 164a facing 4200g and 164b facing 4200h.

The inserter/impactor 4000 holds the disc 160 in a preferred position with respect to the inserter/impactor 4000. (It should be understood that the surfaces of the wedge-shaped extension 4042 can be modified within the scope of the present invention to hold the disc 160 (or another orthopedic device) at positions other than those illustrated herein.) In the illustrated embodiment of the inserter/impactor 4000 in use with the disc 160, the preferred position is with the baseplates 168a,b of the disc 160 angle at 15 degrees of lordosis with respect to one another. More particularly, preferably, the upper and lower surfaces (e.g., 4200g and 4200h) of the wedge-shaped extension 4042 protrude from the distal end 4040 and are formed to hold the baseplates 168a,b such that they are angled at 15 degrees of lordosis with respect to one another. A surface (e.g., lower surface 4200h) of the wedge-shape extension 4042 that mates with an inwardly facing surface of a baseplate (e.g., the lower baseplate 168b) of a disc (e.g., 160) may be correspondingly shaped (e.g., curved or flat) for interaction or mating with the disc baseplate (e.g., the lower surface 4200h of the wedge-shaped extension as illustrated is curved to accommodate the surface of the shield of the disc). Preferably, the forward surface 4200i of the wedge-shaped extension 4042 has a concave curvature towards the shaft 4020 of the inserter/impactor 4000, also for accommodating the curvature of the surface of the shield of the disc.

Also preferably with regard to the preferred positioning, the wedge surfaces of the distal end 4040 protrude from a distance midway with respect to the top and bottom of the distal end 4040 and span (e.g., right to left or vice-versa) the entire distal face of the distal end 4040, and the surfaces 4200d-f above the wedge on the distal end 4040 are respectively perpendicular to the wedge's upper surface 4200g such that each is disposed in parallel with its respective corresponding surface of the disc 160 when the disc 160 is held by the inserter/impactor 4000 at the appropriate lordosis angle. (And, accordingly, are angled approximately 15 degrees with respect to the surfaces below the wedge 4200a-c.) Preferably, for an anterior approach, the wedge-shaped extension 4042 is designed and shaped to fit with its antero-lateral confronting surfaces (4200d,f and 4200a,c) tightly against the correspondingly antero-laterally facing surfaces (180d,f and 180a,c) of the disc 160, but such that its anterior confronting surfaces (4200e and 4200b) are slightly spaced from the anteriorly facing surfaces (180d and 180b) of the disc 160, when the disc is held by the inserter/impactor 4000. This is primarily to address manufacturing issues (in some cases, tolerances may not be adequately defined to ensure that all of those surfaces fit tightly against their corresponding surfaces), so that if there are manufacturing anomalies, any slight tolerance differences that may exist are nevertheless still adequate to ensure at least the tight fitting of the antero-lateral confronting surfaces, so that manipulation of the disc 160 is possible (e.g., in the manner of a wrench against an angled nut). This can be achieved, e.g., by designing the anterior confronting surfaces (4200e and 4200b) to each be slightly greater in length than the corresponding anteriorly facing surfaces (180e and 180b) of the disc baseplates, while still being angled with respect to the antero-lateral confronting surfaces (4200d,f and 4200a,c) at the same angle the antero-laterally facing surfaces (180d,f and 180a,c) of the disc baseplates are angled with respect to the anteriorly facing surfaces (180e and 180b) of the disc. The increased length of the anterior confronting surfaces on the wedge extension results in the slight clearance between the anteriorly facing surfaces (180e and 180b) of the disc and the corresponding anterior confronting surface (4200e and 4200b) of the wedged distal end, thereby ensuring that the disc will be fully seated against the antero-lateral confronting surfaces of the distal end despite possible manufacturing, material or other inevitable variations in tolerances of the artificial intervertebral disc or the inserter/impactor. As noted above, similar in this regard to the manner in which a wrench engages a nut, this fitting increases the mechanical advantage toward repositioning the disc in the intervertebral space. It should be noted, inasmuch as the inserter/impactor 4000 described herein can engage the disc from the antero-lateral angles as well, the anterior confronting surfaces (4200e and 4200b) should also be longer than the antero-laterally facing surfaces (180d,f and 180a,c) of the disc, so that a similar fitting occurs when the disc is held from the antero-lateral angles. Stated broadly, the primary confronting surfaces (e.g., the anterior confronting surfaces) of the inserter/impactor are preferably slightly longer than the primary confronted surfaces (e.g., anteriorly facing surfaces) of the disc for any given holding orientation.

Further, the inserter/impactor 4000 includes a holding pin 4080 that extends from the wedge 4042 along a longitudinal axis of the shaft 4020, the pin 4080 having a distal end 4100 that is bent downwardly. The holding pin 4080 is spring loaded (e.g., by a spring 4090) in a central channel of the shaft 4020, so that it is biased toward the shaft 4020 (preferably, the bent end 4100 of the pin 4080 prevents it from entering the central channel). The holding pin 4080 is restricted from upwardly lateral movement with respect to the distal end of the inserter/impactor by the presence of the wedge-shaped extension 4042 of the distal end 4040 of the inserter/impactor 4000. More particularly, with any attempted upward movement of the holding pin 4080, the pin encounters the upper surface of the channel in which the pin 4080 travels, preventing any such upward movement. The holding pin 4080 is preferably heat treated (e.g., cold formed) to increase material quality (e.g., strength).

A flange 4110, mechanically connected to the pin 4080 and translating adjacent the shaft 4020, can be pushed distally to overcome the bias of the spring 4090 to space the pin 4080 away from the wedge 4042. (An alternative configuration is one in which the flange 4110 and the pin 4080 are formed from a single piece, rather than being mechanically connected.) In this extended position, the pin 4080 can be inserted in a hole (e.g., 182b) in the baseplate (e.g., 168b) of the artificial intervertebral disc (e.g., 160). Releasing the flange 4110 allows the spring 4090 to pull the pin 4080 back, causing the anteriorly facing surface 180b of the baseplate 168b to be held against the lower central flat surface 4200b of the inserter/impactor 4000 and the anterioloaterally facing flat surfaces 180a,c of the artificial intervertebral disc 160 to be held against the other corresponding flat surfaces 4200a,c of the inserter/impactor 4000. This can be further understood in light of the description of the manner in which the inserter/impactor of the '160 application functions to grip an orthopedic device, which is included in the '160 application and incorporated by reference herein. Simultaneously, the anteriorly facing surface 180e of the baseplate 168a is pulled against the upper central flat surface 4200e of the inserter/impactor 4000 and the anterioloaterally facing flat surfaces 180d,f of the artificial intervertebral disc 160 is pulled against the other corresponding flat surfaces 4200d,f of the inserter/impactor 4000. Additionally, the upper and lower wedge surfaces (4200g,h) interfere between the inwardly facing surfaces 164a,b of the disc baseplates, causing the baseplate to be angled at a 15 degree lordosis angle, with the lower surface 164a of the upper baseplate 168a held against the upper surface 4200g, and the upper surface of the shield being held against the lower surface 4200h, as best shown in FIGS. 4a-e.

A knob 4120, threaded on the shaft 4020, can be rotated about the longitudinal axis of the shaft 4020 to push the flange 4110 farther proximally, to pull the pin 4080 tighter and therefore lock its position (the interference of the threads of the knob-shaft interface prevents the knob 4120 from moving distally unless the knob 4120 is reverse rotated to effect that result) to more securely hold the baseplate 168b, and reverse rotated to unlock and loosen the pin 4080.

When the disc 160 is held in this manner, rotation of the disc 160 about a longitudinal axis (of the disc 160) relative to the inserter/impactor 4000 is prevented by interference of the corners of the disc's 160 flat surfaces (180a-c and 180d-f) and the corners of the inserter/impactor's 4000 flat surfaces (4200a-c and 4200d-f), similar to the manner in which a wrench holding a nut prevents rotation of the nut relative to the wrench. Further, the holding of the disc 160 in this manner allows for some repositioning of the disc 160 in the intervertebral space via rotation of the disc 160 in either direction about the longitudinal axis of the intervertebral space. Further when the disc is held in this manner, rotation of the disc about a lateral axis (of the disc 160) relative to the inserter/impactor 4000 is prevented by interference of the inwardly facing surface 164a of the first baseplate (e.g., upper baseplate) of the disc and the upper surface 4200g of the wedge on the distal end 4040, and by interference of the inwardly facing surface 164b of the second baseplate (e.g., lower baseplate) of the disc and the lower surface 4200h of the wedge on the distal end 4040. Accordingly, the holding of the disc in this manner allows for some repositioning of the disc in the intervertebral space via rotation of the disc in either direction about the longitudinal or latitudinal axis of the intervertebral space

In some embodiments, when the artificial intervertebral disc 160 is held by the inserter/impactor 4000, the flat surfaces 180a-c are more closely confronted by the angled flat surfaces 4200a-c of the inserter/impactor 4000, compared with the flat surfaces 180d-f being less closely confronted by the angled flat surfaces 4200d-f of the inserter/impactor 4000. As such, the structure of the artificial intervertebral disc 160 having the flat surfaces 180d-f (e.g., the upper baseplate 168a) has slightly more rotation and angulation freedom relative to the inserter/impactor 4000 when being held, compared to the structure of the artificial intervertebral disc 160 having the flat surfaces 180a-c (e.g., the lower baseplate 168b). This permits the artificial intervertebral disc 160 to adjust to the intervertebral space (e.g., to the angulation of the adjacent vertebral endplates, defining the intervertebral space, relative to one another) as it is being inserted thereinto. That is, typically, the adjacent vertebral endplates will be lordotically angled with respect to one another as a result of the intervertebral space being prepared and distracted.

Preferably, in order to provide for a holding of the disc 160 for two additional (here, anteriolateral) insertion approaches, each disc 160 also includes two additional holes 182a and 182c, one (e.g., 182a) spaced apart from one of the anteriolaterally facing flat surfaces (e.g. 180a), and the other (e.g. 182c) spaced apart from the other of the anteriolaterally facing flat surfaces (e.g. 180c). Accordingly, operation of the inserter/impactor 4000 can fit the holding pin 4080 into either of these two additional holes 182a or 182c, and hold the associated anteriolaterally facing flat surface (the one associated with the hole into which the pin 4080 is fit) of the disc 160 against the flat surface of the inserter/impactor 4000 opposite the pin 4080. For example, in a first anteriolateral approach for the disc 160, 180a and 180d facing 4200b and 4200e, 180c and 180f not confronted, and 180b and 180e facing 4200c and 4200f, and in a second anteriolateral approach for the disc 160, 180b and 180e facing 4200a and 4200d, 180a and 180d not confronted, and 180c and 180f facing 4200b and 4200e. It should be understood that preferably, in order to facilitate these additional approaches, the angle separating the anteriorly facing flat surface of the disc 160 and one of the anteriolaterally facing flat surfaces of the disc 160 is equal to the angle separating the anteriorly facing flat surface and the other of the anteriolaterally facing flat surfaces. Preferably, the surfaces are angled with respect to one another at an angle of 33.4 degrees.

It should also be understood that the inclusion of additional adjacent angulated surfaces (or placing the angulated surfaces in other locations on the disc or other orthopedic device), and/or including corresponding holes adjacent to such surfaces, can provide the surgeon with additional approaches, e.g., other anteriolateral approaches, directly lateral approaches, posteriolateral approaches, and/or directly posterior approaches. For example, a trial or disc can have angled surfaces (and corresponding holes) along the entire perimeter of one or both of the baseplates, and thus enable the surgeon to engage the trial or disc from a number of angles, including anterior, posterior, lateral, anteriolateral, and posteriolateral angles.

The inserter/impactor 4000 further includes at a proximal end a cap 4140 for use as an impact surface if the disc 160 must be impacted further into the intervertebral space after insertion, or forcibly extracted from the intervertebral space. A mallet can be used to strike the cap 4140 (in a distal direction for impaction, or in a proximal direction (using the flange of the cap 4140) for extraction). It should be noted a striking of the cap 4140 will translate the striking force to the baseplates through the shaft 4020 and the flat surfaces, but will not damage the holding pin 4080 because the holding pin 4080 is spring loaded in the central channel and thus buffered from the striking force thereby. The distal end 4040 of the inserter/impactor 4000 further preferably includes at least one vertebral body stop 4202 that protrudes longitudinally with respect to the shaft 4020, from the surfaces of the distal end. The stops help prevent the inserter/impactor from being used to insert the disc (or other orthopedic device) too far into the intervertebral space.

Accordingly, the inserter/impactor 4000 can be used to grip the artificial intervertebral disc to be implanted, and hold the same during insertion and/or removal of the same, and is useful for a variety of surgical approach angles.

While there has been described and illustrated specific embodiments of instrumentation, it will be apparent to those skilled in the art that variations and modifications are possible without deviating from the broad spirit and principle of the invention. The invention, therefore, shall not be limited to the specific embodiments discussed herein.

1. A system for stabilizing an intervertebral segment comprising:
an intervertebral implant including
a first plate,
a second plate, and
an articulating joint coupling said first and second plates, wherein at least one of said plates has an inner surface having at least one hole;
an instrument for holding said implant including
a shaft having a proximal end, a distal end and a longitudinal axis extending between the proximal and distal ends,
a body attached to the distal end of said shaft,
a pin having a hooked end that is extendable from said body for being inserted into the at least one hole of said implant for coupling said implant with the distal end of said holding instrument.

2. The system as claimed in claim 1, wherein said pin is movable along the longitudinal axis between a first position in which said hooked end of said pin is at least partially retracted inside said body and a second position in which said hooked end of said pin is at least partially extended from said body.

3. The system as claimed in claim 1, further comprising a spring coupled with said pin for normally urging said pin into the first retracted position.

4. The system as claimed in claim 1, further comprising a flange coupled with said pin, said flange being movable relative to said shaft and toward the distal end of said shaft for moving said hooked end of said pin into the second extended position.

5. The system as claimed in claim 1, wherein said pin has a first section that extends along said longitudinal axis of said shaft and said hooked end is connected to and angled relative to said first section of said pin.

6. The system as claimed in claim 1, wherein said hooked end of said pin consists of a single hook.

7. The system as claimed in claim 1, wherein said body has at least one concave surface at a distal face thereof that is engageable with said intervertebral implant.

8. The system as claimed in claim 7, wherein said at least one concave surface comprises:
a first set of substantially flat surfaces that are angled relative to one another;
a second set of substantially flat surfaces that are angled relative to one another;
a wedge-shaped projection located between said first set of substantially flat surfaces that are angled relative to one another and said second set of substantially flat surfaces that are angled relative to one another.

9. The system as claimed in claim 8, wherein said first plate has a peripheral edge including substantially flat surfaces that are angled relative to one another and that conform with said first set of substantially flat surfaces on said body and said second plate has a peripheral edge including substantially flat surfaces that are angled relative to one another and that conform with said second set of substantially flat surfaces on said body.

10. The system as claimed in claim 8, wherein said wedge-shaped projection has a top surface, a bottom surface angled relative to the top surface and a curved surface extending between the top and bottom surfaces thereof.

11. The system as claimed in claim 11, wherein said wedge-shaped projection includes a channel formed therein and said hooked end of said pin is extendable from said channel.

12. A system for stabilizing an intervertebral segment comprising:
an articulating implant including a first plate and a second plate, wherein an inner surface of at least one of said plates has at least one hole;
an instrument for holding said implant including
a shaft having a proximal end and a distal end,
a body attached to the distal end of said shaft,
a pin having a hooked end that is extendable from said body and insertable into the at least one hole of said implant for coupling said implant with said holding instrument.

13. The system as claimed in claim 12, wherein said pin is movable along an axis extending from the proximal end to the distal end of said shaft between a first position in which said hooked end of said pin is at least partially retracted inside said body and a second position in which said hooked end of said pin is at least partially extended from said body.

14. The system as claimed in claim 13, further comprising a spring coupled with said pin for normally urging said hooked end of said pin toward the proximal end of said shaft and into the first position.

15. The system as claimed in claim 12, wherein said body has at least one concave surface at a distal end thereof that is engageable with said intervertebral implant.

16. The system as claimed in claim 15, wherein said at least one concave surface comprises:
a first set of substantially flat surfaces that are angled relative to one another;
a second set of substantially flat surfaces that are angled relative to one another;
a wedge-shaped projection located between said first set of substantially flat surfaces and said second set of substantially flat surfaces.

17. The system as claimed in claim 16, wherein said first plate has a peripheral edge including substantially flat surfaces that are angled relative to one another and that conform with said first set of substantially flat surfaces on said body and said second plate has a peripheral edge including substantially flat surfaces that are angled relative to one another and that conform with said second set of substantially flat surfaces on said body.

18. A system for stabilizing an intervertebral segment comprising:
an articulating implant including a first plate and a second plate, wherein an inner surface of at least one of said plates has at least one hole;
an instrument for holding said implant including
a shaft having a proximal end and a distal end,
a body attached to the distal end of said shaft,
a distal face of said body including a first set of substantially flat surfaces that are angled relative to one another, a second set of substantially flat surfaces that are angled relative to one another, and a wedge-shaped projection located between said first set of substantially flat surfaces and said second set of substantially flat surfaces, and
a pin having a hooked end that is extendable from said wedge-shaped projection for being insertable into the at least one hole of said implant for coupling said implant with said holding instrument.

19. The system as claimed in claim 18, wherein said wedge-shaped projection has a top surface engageable with said first plate, a bottom surface engageable with said second plate, the top surface of said wedge-shaped projection being angled relative to the bottom surface of said wedge-shaped projection, and a concave surface extending between the top and bottom surfaces thereof.

20. The system as claimed in claim 19, wherein said wedge-shaped projection includes a channel formed therein and said hooked end of said pin is extendable from said channel.
